(19) United States
(12) Patent Application Publication
(10) Pub. No.: US 2014/0038211 A1
(43) Pub. Date: Feb. 6, 2014

(54) BREAST CANCER DIAGNOSIS USING NIPPLE DISCHARGE

(71) Applicant: Edward R. Sauter, Tyler, TX (US)

(72) Inventor: Edward R. Sauter, Tyler, TX (US)

(73) Assignee: University of North Dakota, Grand Forks, ND (US)

(21) Appl. No.: 13/984,217

(22) PCT Filed: Jan. 26, 2012
(86) PCT No.: PCT/US12/22754

§ 371 Date: Oct. 21, 2013

Related U.S. Application Data
(60) Provisional application No. 61/441,452, filed on Feb. 10, 2011.

(51) Int. Cl. G01N 33/68

(52) U.S. Cl. CPC G01N 33/6893
USPC 435/7.92

(57) ABSTRACT

A panel of biomarkers for the prediction of breast cancer using nipple discharge, as well as method and kits for using the same. Nipple discharge may be nipple aspirate fluid or spontaneous discharge. The panel has been selected to optimize predictive ability while minimizing the number of markers to be screened. As observed, the panel approaches one hundred percent accuracy for predicting breast cancer without the need for invasive biopsy.

### CROSS-REFERENCE TO RELATED APPLICATIONS

This application claims priority from U.S. provisional patent application Ser. No. 61/441,452, filed Feb. 10, 2011.

### STATEMENT REGARDING FEDERALLY SPONSORED RESEARCH

This invention was made with government support under Grant No. CA119095 awarded by the National Institutes of Health. The government has certain rights in the invention.

### BACKGROUND OF THE INVENTION

1. Field of the Invention

The present invention generally relates to the detection of a panel of carbohydrate and protein biomarkers present in nipple discharge and use of the same to diagnose breast cancer prior to, or in lieu of, invasive breast biopsy.

2. Background Art

Early detection of breast cancer, aided by reliable predictive methodologies, facilitates successful disease treatment and management. Early detection is a major factor contributing to the 2.3% annual decline in breast cancer death rates over the past 10 years. Nonetheless, in 2009, 40,170 women in the U.S. died from breast cancer. Additionally, there are 2.5 million breast cancer survivors in the U.S. who remain at increased risk for a second breast cancer. When an invasive biopsy is performed to exclude cancer, some 66-85% of these interventions find only benign disease.

Currently the best early detection approaches are based on imaging, but the quantification of biochemical markers in tissues and/or fluids accessible via less or non-invasive methods is an attractive alternative. Such approaches have the potential to be cheaper, faster, safer and more widely adopted; further, if more appropriate biomarkers and detection methods can be identified, they promise to offer greater specificity and sensitivity, respectively. Although there has been considerable effort in the discovery arena targeted at identifying novel biomarkers and/or combinations thereof that might ultimately offer clinical utility, few candidates have been subjected to rigorous verification and/or validation.

Recently, nipple aspiration of breast fluid has shown promise in assisting cost-effective and non-invasive detection of in situ and invasive breast cancer. A type of nipple discharge, nipple aspirate fluid (NAF) can be obtained from up to 95% of women (Sauter et al. (1997) Br Cancer 76: 494-501), noninvasively and at low cost. NAF further contains relatively high levels of proteins, carbohydrates and lipids secreted from ductal and lobular epithelia, but only a small number of cancer cells in those patients with cancer (Glinsky (2001) Cancer Research 61: 4851-4857; Hsiung et al., Cancer Journal (2002) 8, 303-310).

The Thomsen-Friedenreich (TF; Galactose-β-(1→3)-N-acetyl-D-galactosamine) antigen is found in breast carcinoma but not in healthy breast tissue (Springer et al. (1980) Cancer 45: 2949-29541). It has been reported that TF is displayed on cell-surface proteins and lipids in 70% to 90% of adenocarcinomas of the breast (Springer (1984) Science 224: 1198-1206). TF is upregulated in the nipple discharge fluid (which includes NAF as well as spontaneous nipple discharge which can be physiologic or pathologic) of post-menopausal (but not pre-menopausal) women with breast atypia and cancer, and correctly classifies either cancer or abnormal vs. benign pathology 83% of the time in post-menopausal women (Deutscher et al. (2010) BMC Cancer 10: 519).

In certain studies, greater than 85% of patients with ductal breast cancer were TF antigen positive while over 94% of patients with benign breast disease were negative (Springer et al. (1980) Cancer 45: 2949-29541; Springer (1984) Science 224: 1198-1206). TF is an early differentiation carbohydrate antigen that is linked to Ser/Thr on glycoproteins and can be found on cancer-associated glycolipids and ceramides. TF antigen is covalently masked in healthy individuals but exposed and immunoreactive in greater than 90% of carcinoma patients. It has been reported that there is a positive correlation between the amount of TF antigen and the carcinoma's aggressiveness (Springer, (1984) Science 224: 1198-1206; Glinsky, (2001) Cancer Research 61: 4851-4857). It is also known that the TF antigen, present on the surface of breast carcinoma cells, plays an important role in the early stages of metastatic deposition (Glinsky (2001) Cancer Research 61: 4851-4857). TF antigen was found to be elevated in the nipple aspirate fluid of women requiring diagnostic breast biopsy who, in fact, were found to have breast atypia and cancer. Deutscher (2010) BMC Cancer 10: 519-526.

The TF antigen can be detected via the galactose oxidase-Schiff (GOS) reaction. The GOS reaction yields positive results in many malignancies, including carcinomas of the breast, as well as the lung, pancreas, ovary, thyroid, stomach, and colon. This reaction has been studied in breast tissue sections and has been reported to yield positive results in breast carcinoma tissue and negative results in normal breast tissue, using a spectrophotometric assay system (Shamsuddin (1995) Cancer Res 55: 149-152).

Cancer cell invasion and metastasis requires the degradation of the extracellular matrix (ECM) and basement membrane. This process is accomplished by several proteins, including those of the plasminogen activator (PA) system. For example, urokinase-type plasminogen activator (uPA) and plasminogen activator inhibitor (PAI)-1 are proteins involved in basement membrane degradation. (Qin et al. (2003) Ann Surg Oncol 10: 948-953). In fact, uPA plays a key role in ECM degradation. In women with breast cancer, uPA appears to promote cancer invasion and metastasis through degradation of the ECM, stimulation of angiogenesis, alteration in cell migration and adhesion, and inhibition of apoptosis (Duffy (2002) Clin Chem 48: 1194-7; Andreasen et al. (1997) Int J Cancer 72: 1-22; Ma et al. (2001) J Cell Sci 114:3387-96).

Plasminogen activator activity is inhibited by PAI-1 (Blasi (1999) Thromb Haemost 82: 298-304). PAI-1 promotes breast cancer invasion and metastasis. Deficient PAI-1 expression in mice prevented local invasion and tumor vascularization of transplanted malignant keratinocytes. When PAI-1 expression was restored, invasion and associated angiogenesis were also restored, suggesting that host-produced PAI-1 is essential for cancer cell invasion and angiogenesis (Bajou et al. (1998) Nat Med 4: 923-8). PAI-1 promotes angiogenesis by directly inhibiting proteases, suggesting that excessive plasmin proteolysis may prevent the assembly of tumor blood vessels (Bajou et al. (2001) J Cell Biol 152: 777-84). Possible mechanisms by which PAI-1 promotes breast cancer include prevention of excess ECM degradation, modulation of cell adhesion, a role in angiogenesis, and the stimulation of cell proliferation (Duffy (2002) Clin Chem 48: 1194-7). The association of uPA and PAI-1 expression with breast cancer is complex.

In a pooled analysis of 8377 breast cancer patients, higher uPA and PAI-1 levels in tumor tissue were found to directly relate to worse prognosis (Look et al. (2002) J Natl Cancer Inst 94: 116-28). It has been reported that both uPA and PAI-1 are useful in predicting breast cancer (Qin et al. (2003) Cancer J 9: 293-301). Specifically, high levels in NAF of uPA and PAI-1 significantly contributed to a model that predicted which women had breast cancer. uPA and PAI-1 are concentrated in NAF compared to plasma (Id.) Both types of nipple discharge can be obtained non-invasively and contain concentrated secreted proteins, carbohydrates and lipids from the breast ductal epithelium, the cells that give rise to cancer.

However, a need exists for a reliable method to predict the presence or absence of breast cancer in human subjects suspected of havinge cancer prior to confirmation of presence or absence of the cancer by biopsy or surgery.

### SUMMARY OF THE INVENTION

The present invention is generally directed to an assay allowing determination of a panel of carbohydrate and protein biomarkers present in nipple discharge (ND). Persons skilled in the art will appreciate that the term nipple discharge includes both nipple aspirate fluid (NAF) and spontaneous nipple discharges such as pathologic nipple discharge (PND). The biomarkers of interest occur on protein, carbohydrate, and lipid molecules present in ND and can serve as indicators and/or predictors of breast cancer. The methods described herein facilitate the non-invasive detection of breast cancer at a variety of stages, and serve as a diagnostic method or a “predictive measure” that can signal the presence of cancer (positive prediction) or absence of cancer (negative prediction).

One aspect of the invention is a novel panel of biomarkers, preferably to include TF, uPA and PAI-1. The relative levels of these biomarkers are highly indicative of the presence of breast cancer or precancer in a human, e.g., a man or woman requiring breast biopsy to exclude disease and so are “predictive” of the subsequent biopsy/histology results. These biomarkers are capable of reliable measurement in the samples collected as disclosed hereinbelow. When TF, uPA and PAI-1 were all employed, their predictive ability approached 100% in both pre- and post-menopausal women requiring breast biopsy to exclude disease (Table 2, FIG. 1D). Two of the ND biomarkers (TF and PAI-1) are more predictive in post- than premenopausal women, whereas uPA is more predictive in premenopausal women. The markers are highly predictive of disease, regardless of whether the disease is precancer or cancer (“atypia”) and whether or not pathologic nipple discharge (PND) was present.

Thus, the present invention provides a method for diagnosing breast cancer in a human subject suspected of having breast cancer, comprising the steps of: collecting nipple discharge from said subject; analyzing said discharge for the level of (a) the TF carbohydrate biomarker, (b) the uPA protein biomarker and (c) the PAI-1 protein biomarker; and determining the presence or absence of breast cancer based on the results of said analysis.

In another embodiment, the present invention provides a method for diagnosing breast cancer in a human subject suspected of having breast cancer comprising the steps of: (i) analyzing a sample of nipple discharge from said subject to determine the level of (a) the TF carbohydrate biomarker, (b) the uPA protein biomarker and (c) the PAI-1 protein biomarker; (ii) determining the presense or absence of breast cancer based on the results of the analysis; and (iii) informing the subject or his/her physician of the presence or absence of breast cancer.

Another aspect of the present invention is a kit for detection of these biomarkers, which employs the techniques of immunoassay, e.g., of ELISA, to measure the levels of the three biomarkers.

One embodiment of the kit for detection of carbohydrate and protein biomarkers in nipple discharge can comprise:

(a) a capture agent that binds specifically to TF;

(b) a capture agent that binds specifically to uPA;

(c) a capture agent that binds specifically to PAI-1;

each preferably immobilized on an inert substrate; and

(d) a labeled binding agent that binds to bound TF;

(e) a labeled binding agent that binds to bound uPA;

(f) a labeled binding agent that binds to bound PAI-1; and

(g) instructions for use of the kit in a method according to claim 1 or 2.

In the embodiment in which the labeled binding agents are monoclonal antibodies that comprise labels such as an enzyme or a binding site for an enzyme, the kit can also include the substrate for the enzyme, or both an enzyme that can bind to the binding site and its substrate, as described hereinbelow.

Other objects and features will be in part apparent and in part pointed out hereinafter.

### SUMMARY OF THE FIGURE

FIG. 1. ROC curves for the prediction of breast cancer. TF, uPA, PAI-1 and age information were used to generate curves comparing cancer=DCIS and invasive cancer vs. no cancer=normal, hyperplasia and atypical hyperplasia: TF and age in post-menopausal women (AUC=0.81) (A), uPA and age in pre-menopausal women (AUC=0.87) (B), TF, uPA and age in all (pre- and post-menopausal) women (AUC=0.84) (C), and TF, uPA, PAI01 and age in all women (AUC=1.0) (D).

### DETAILED DESCRIPTION OF THE INVENTION

### List of Abbreviations

The term “presence” of the biomarkers includes absolute and relative levels as well as presence or absence thereof. Abs: absorbance; ADH: atypical ductal hyperplasia; CV: coefficient of variation; DCIS: ductal carcinoma in situ; H: usual hyperplasia; ICC: intraclass correlation coefficient; NAF: nipple aspirate fluid; ND: nipple discharge; OR: odds ratio; P: pathologic; ROC: receiver operating characteristic; “suspected of having breast cancer:” a woman exhibiting breast tissue lesions as described herein below so that breast biopsy is indicated to exclude cancer. “Cancer” is defined herein as pathologic evidence of either ductal carcinoma in situ (DCIS) or invasive cancer; “no cancer” is defined herein as histopathology which was normal, usual hyperplasia, and/or atypical hyperplasia; “benign” is defined herein as histopathology containing normal findings and/or usual hyperplasia; and “abnormal” is defined herein as histopatology demonstrating atypical hyperplasia, DCIS, and/or invasive cancer.

The present invention is generally directed to an assay allowing determination of a panel of carbohydrate and protein biomarkers that occur on protein, carbohydrate, and lipid molecules present in ND. In several embodiments, the carbohydrate and protein biomarkers are indicators and predictors of breast cancer. In a preferred embodiment, the panel of biomarkers is selected for optimal reliability at predicting breast cancer. In this context, optimal reliability is characterized by minimizing the number of markers while still yielding statistically significant results and minimizing false positives as well as false negatives. The methods described herein facilitate the non-invasive detection of breast cancer at a variety of stages, and serve as a predictive measure that can signal an increased chance of developing cancer, or that is indicative of the presence of cancer or the absence of cancer in a subject who does not exhibit abnormalities suggestive of, or that give rise to the suspicion of the presence of breast cancer.

Generally, detection of the levels carbohydrate biomarkers is accomplished via an assay having the sensitivity necessary to detect low levels of Thomsen-Friedenreich (TF) carbohydrate biomarker in ND. For example, the capture assay described in US Pat App. Pub. No. 2008/0293161 by Deutscher et al., incorporated herein by reference, employs a TF-specific capture agent to isolate, from the ND sample, proteins, lipids, or carbohydrates displaying a TF carbohydrate biomarker upon their surface.

Deutscher et al. '161 describe both indirect and direct detection means. In indirect detection, a tagged binding agent binds to unoccupied capture agent binding sites. Quantitation of the amount of tag present correlates, inversely (i.e., indirectly), to the amount of TF carbohydrate biomarker captured from the ND sample. In direct detection, the tagged binding agent such as a biotinylated monoclonal antibody (MCA) specific for TF, binds directly to TF, which in turn is bound to the immobilized capture agent. Quantitation of the amount of tag present correlates directly with the amount of TF carbohydrate biomarker captured from the ND sample. For example, the biotin tag can be reacted with an avidin-labelled enzyme and the amount of breast protein or carbohydrate quantified by reaction with its substrate, as discussed hereinbelow. Either detection means may be employed; direct detection means are preferred.

The TF carbohydrate biomarker assay described by Deutscher et al. (2010) can be employed to detect breast cancer at several different stages. Persons skilled in the art will recognize the following stages as being representative:
Ductal carcinoma in situ (DCIS);
Stage IA (T1, N0, M0);
Stage IB (T0 or T1, N1mi, M0);
Stage IIA—One of the following applies:
   T0 or T1, N1 (but not Nlmi), M0: the tumor is 2 cm or less across (or is not found) (T1 or T0) and either it has spread to 1 to 3 axillary lymph nodes, with the cancer in the lymph nodes larger than 2 mm across (N1a) or tiny amounts of cancer are found in internal mammary lymph nodes on sentinel lymph node biopsy (Nib) or it has spread to 1 to 3 lymph nodes under the arm and to internal mammary lymph nodes (found on sentinel lymph node biopsy) (N1c);
   OR
   T2, N0, M0: The tumor is larger than 2 cm across and less than 5 cm (T2) but hasn't spread to the lymph nodes (N0), and the cancer hasn't spread to distant sites (M0);
Stage IIB—One of the following applies:
   T2, N1, M0: The tumor is larger than 2 cm and less than 5 cm across (T2) and has spread to 1 to 3 axillary lymph nodes and/or tiny amounts of cancer are found in internal mammary lymph nodes on sentinel lymph node biopsy (N1), and the cancer hasn't spread to distant sites (M0);
   OR
   T3, N0, M0: The tumor is larger than 5 cm across but does not grow into the chest wall or skin and has not spread to lymph nodes (T3, N0), and the cancer hasn't spread to distant sites (M0);
Stage IIIA—One of the following applies:
   T0 to T2, N2, M0: The tumor is not more than 5 cm across (or cannot be found) (T0 to T2). It has spread to 4 to 9 axillary lymph nodes, or it has enlarged the internal mammary lymph nodes (N2). The cancer hasn't spread to distant sites (M0);
   OR
   T3, N1 or N2, M0: The tumor is larger than 5 cm across but does not grow into the chest wall or skin (T3). It has spread to 1 to 9 axillary nodes, or to internal mammary nodes (N1 or N2). The cancer hasn't spread to distant sites (M0);
Stage IIIB—T4, N0 to N2, M0: The tumor has grown into the chest wall or skin (T4), the cancer hasn't spread to distant sites (M0), and one of the following applies:
   It has not spread to the lymph nodes (N0);
   It has spread to 1 to 3 axillary lymph nodes and/or tiny amounts of cancer are found in internal mammary lymph nodes on sentinel lymph node biopsy (N1);
   It has spread to 4 to 9 axillary lymph nodes, or it has enlarged the internal mammary lymph nodes (N2);
Stage IIIC—any T, N3, M0: The tumor is any size (or can't be found), the cancer hasn't spread to distant sites (M0), and one of the following applies:
   Cancer has spread to 10 or more axillary lymph nodes (N3).
   Cancer has spread to the lymph nodes under the clavicle (collar bone) (N3).
   Cancer has spread to the lymph nodes above the clavicle (N3).
   Cancer involves axillary lymph nodes and has enlarged the internal mammary lymph nodes (N3).
   Cancer has spread to 4 or more axillary lymph nodes, and tiny amounts of cancer are found in internal mammary lymph nodes on sentinel lymph node biopsy (N3).
Stage IV—any T, any N, M1: The cancer can be any size (any T) and may or may not have spread to nearby lymph nodes (any N); it has also spread to distant organs or to lymph nodes far from the breast (M1).

This TF carbohydrate biomarker assay can also be employed to detect atypical ductal hyperplasia (ADH), a condition considered as a risk factor for developing cancer where abnormal cells are present. When used to detect atypical hyperplasia, the presence of TF carbohydrate biomarker would signal an increased chance of developing cancer.

uPA and PAI-1 can also be assayed using enzyme-linked immunosorbent assays (ELISA). ELISA kits for uPA and PAI-1 are available from American Diagnostica, Inc. (Greenwich, Conn.). Levels of these two markers are determined according to the manufacturer's instructions.

### Collection of Nipple Discharge

Nipple discharge includes aspirate fluid as well as spontaneous discharge, and either can be obtained through a variety of methods known in the art (see, e.g., Sauter et al. (1997) Br J Cancer 76: 494-501). Nipple discharge bathes the ductal epithelial cells, which undergo malignant transformation in most forms of breast cancer. The fluid contains exfoliated ductal epithelial cells, proteins, and lipids secreted from the ductal and lobular epithelia. Samples can be collected noninvasively, for example, by using a modified breast pump and/or by manual massage and expression.

Various procedures can facilitate sample collection. These include warming the breast with, for example, a warm moist cloth or heating pad, and/or massage of the breast before, during, and/or after collection. Collection facilitated by massage can occur by manually expressing the breasts by placing hands flat around the base of the breast and squeezing down toward the tip of the nipple. During collection, keratin plugs can block aspiration. Dekeratinizing the nipple can be performed with rough gauze along with alcohol or Cerumenex 3% (Triethanol polypeptide).

Nipple aspirate fluid is collected noninvasively using a modified breast pump. The nipple is cleansed with alcohol. A warm, moist cloth is placed on the breast after the alcohol evaporates. The cloth is removed after 2 minutes. While the breast is massaged, a syringe connected to the breast pump collected NAF. Aspiration is repeated on the opposite breast, if present. Spontaneous nipple discharge is collected directly from the breast, generally without the use of a pump. Fluid, either NAF or spontaneous nipple discharge, in the form of droplets (1-200 μl) is collected in capillary tubes, and the samples are preferably immediately snap frozen at −80° C.

Nipple discharge may be diluted if the sample obtained is a small volume or the sample obtained is particularly viscous. ND may also be diluted to standardize the sample obtained with other samples on a particular parameter. A ND sample may be diluted according to any of a number of known means, including the addition of an inert fluid, such as distilled water or a buffer such as PBS. Alternatively, ND may be concentrated by the removal of water if the volume of the sample is greater than desired for the assay. Water may be removed from a ND sample according to any of a number of known means, including use of a Savant SpeedVac®, lyophilization, or other means which do not remove, in addition to the water, a fraction of the ND which may contain the desired biomarkers (e.g., the lipid fraction, protein fraction, carbohydrate fraction, or cellular debris).

Although generally less preferred, in one embodiment the ND may be fractionated to provide a nipple discharge derivative that may then be analyzed for the desired biomarkers. Fractionation of a ND sample may be accomplished by any of a number of known means, including centrifugation, ultrafiltration, chromatography, gel electrophoresis, and distillation. Thus, for example, a fraction may be obtained that, relative to ND as obtained from a patient (i.e., “complete” or “total” ND), contains a ratio of lipid to protein, lipid to carbohydrate, or protein to carbohydrate that differs from the original sample.

Similarly, the ND may be concentrated, resulting in a concentrate that, relative to ND as obtained from a patient (i.e., “complete” or “total” ND), contains a ratio of lipid to protein, lipid to carbohydrate, or protein to carbohydrate that differs from the original sample. In addition to the alteration of one or more of these ratios, the concentrate may also contain a decreased amount of water relative to ND as obtained from a patient. Such a concentration of a ND sample may be accomplished by any of a number of known means, including, for example, centrifugation and spin filtering, ultrafiltration, chromatography, ammonium sulfate precipitation, TCA/DOC, and gel electrophoresis.

### TF Assay

The assay described by Deutscher et al. (2010) uses a capture agent to select out carbohydrates, proteins, or lipids displaying carbohydrate biomarkers—including TF from the ND sample. This assay can be conducted using any procedure selected from the variety of standard assay protocols generally known in the art. As it is generally understood, the assay is constructed so as to rely on the interaction of the capture agent(s), TF in the sample, and labeled binding agent(s). In both indirect and direct detection methodologies, the reaction can be quantitated by comparing against a standard curve derived from a known amount(s) of non-tagged TF-displaying molecules.

The capture agent of the assay described by Deutscher et al. (2010), such as a MCA specific for TF, is immobilized on a carrier and then exposed to the ND sample, from which the capture agent binds TF if present. A TF capture agent coating a solid phase material will generally bind a sufficient quantity of TF antigen, respectively, within a relatively short period of time (approximately two to five minutes), and retain the captured TF antigen during subsequent washing and detection of labeled binding agent. The density of the capture agent on the carrier can be, for example, from about 200 ng cm⁻² to about 650 ng cm⁻². The amount of capture agent immobilized on the carrier should be in excess of the expected amount of TF in the sample. Generally, TF concentrations in undiluted cancerous ND samples range from about 15 ng/μl to about 2,500 ng/μl. Calculating the amount of capture agent to be immobilized on the carrier as a function of the expected concentration of carbohydrate antigen in the ND and the volume of sample delivered is well within the skill in the art.

Capture agents include immunoglobulin peptides, lectins, bacteriophages, or other polypeptides that bind specifically to TF antigen. Examples of TF-specific lectin capture agents include Amaranthus caudatus lectin; Artocarpus integrifolia Jacalin lectin; Arachis hypogea peanut lectin; Bauhinia purpurea agglutinin; and bBacteriophage displaying TF-binding amino acid peptide (p-30) Immunoglobulin peptide capture agents include, for example, polyclonal antibodies, monoclonal antibodies, and antibody fragments such as proteolytically cleaved antibody fragments and single chain Fv antibody fragments, as further discussed below. It should be understood that theses capture agents do not limit the extent and variety of antibodies that can be used for practicing the methods described herein.

The ND sample solution, or dilutions thereof, is then applied to the capture agent-coated carrier under conditions in which the capture agent binds molecules that display the carbohydrate biomarker of interest. ND can be applied, for example, at the concentration collected from the patient, or serially diluted by, for example, 1/10, 1/50, 1/100, 1/500, or 1/1000. The volume of ND supplied should be such that the amount of immobilized capture agent on the carrier is in excess to the expected amount of TF in the sample, as described above. Suitable conditions are, for example, incubation of 100 μl of diluted nipple aspirate sample for about four hours at room temperature. After allowing sufficient time for binding of carbohydrate biomarker(s) to the capture agent(s), the ND sample is then washed away.

After forming the biomarker-capture agent complex, the carrier is combined with a labeled binding agent. The target of the labeled binding agent will depend upon whether indirect or direct detection means are employed.

In indirect detection assays, the resulting biomarker-capture agent complex is further reacted with a binding agent that has affinity for the capture agent, where the binding agent is attached to an easily assayable tag. In direct detection assays, the resulting biomarker-capture agent complex is further reacted with a binding agent that has affinity for the carbohydrate biomarker, where the binding agent is attached to an easily assayable tag.

The assayable tag may be detectable directly or may bind to a reporter for which it has specificity. The assayable tag attached to the binding agent can be, for example, an enzyme, a coenzyme, an enzyme substrate, an enzyme co-factor, an enzyme inhibitor, a radionuclide, a chromogen, a fluorescer, a chemoluminescer, a free radical, or a dye. Preferably, the tag is biotin, which is then recognized by avidin or streptavidin conjugated to a reporter, such as the enzyme horseradish peroxidase. Alternatively, detection can be mediated by reporter reagents such as fluorescent avidins, streptavidins or other biotin-binding proteins or enzyme-conjugated streptavidins plus a fluorogenic, chromogenic, or chemiluminescent substrate.

Detection follows washing away unbound labeled binding agent. In the indirect detection approach, the tag in the complex formed from the capture agent and tagged binding agent is detected, thereby indirectly indicating the amount of TF present. Thus, for indirect detection methods, when the carbohydrate biomarker of interest is present in the ND sample, low signal will be detected from the label as there will have been fewer available sites for the labeled binding agent to bind.

Alternatively, in the direct detection approach, the tag in the complex formed from the capture agent, carbohydrate biomarker of interest, and the tagged binding agent is detected, thereby directly indicating the amount of TF present. Thus, for direct detection methods, when the carbohydrate biomarker of interest is present in the ND sample, high signal will be detected from the label as there will have been more sites for the labeled binding agent to bind.

Detection methodology will depend upon the identity of the assayable tag on the binding agent, as commonly understood in the art. Kits for detection of tagged binding agents in the capture immunoassay described above are commercially available. Detection procedures include Western blots, enzyme-linked immunosorbent assays, radioimmunoassays, competition immunoassays, dual antibody sandwich assays, immunohistochemical staining assays, agglutination assays, and fluorescent immunoassays.

Preferably, a streptavidin/peroxidase complex is used to assay the amount of biotin tag. The activity of the peroxidase enzyme linked to the streptavidin can then be detected through the addition of a peroxidase substrate such as 2,2′-Azino-bis(3-ethyl benzthiazoline-6-sulfonic acid) (ABTS).

Solutions with known amounts of carbohydrate biomarkers can be used in the generation of standard curves. Examples of TF-displaying molecules that can be used as standards for determining the concentration of TF in ND samples include: Asialofetuin (Sigma Chemical Co., St. Louis, Mo.); Asiaolimucin (Sigma Chemical Co., St. Louis, Mo.); Asialoglycophorin (Sigma Chemical Co., St. Louis, Mo.) and Gal beta 1,3GalNAc-alpha-O-benzyl (Sata et al. (1990) J Histochem Cytochem. 38, 763).

In an exemplary embodiment, TF antigen in ND samples is quantitated by an antigen capture immunoassay as follows. Microtiter wells (Immunomaxi, Switzerland) are coated with 50 μl of anti-TF antibody A78-G/A7 (1 μg/ml in 0.1 M carbonate buffer, pH 9.6) by incubating the plates at 37° C., for 4 hours. After removing the excess antibody, the wells are blocked with 2% BSA in 10 mM Tris-HCl buffer overnight at 4° C. in a humid chamber. After overnight incubation, the plate wells are washed three times with Tris buffered saline containing 0.1% Tween-20 (TTBS), using an automatic plate washer (E1x405, BIO-TEK). To the washed wells, 100 μl of appropriately diluted control and cancer ND samples are added and incubated for 4 hours at room temperature (RT). After washing the wells, 50 μl of biotinylated asialofetuin (ASF) (2.0 mg/ml) is added to the wells and incubated for 1 hour at RT. Unbound ASF is then washed and 100 μl of 1/2000 streptavidin/peroxidase complex (Sigma) in TTBS buffer is added and incubated at RT for 45-60 minutes. Peroxidase activity is demonstrated by incubation in ABTS (2,2-Azinbis(3-ethylbenzthiazoline-6-sulfonic acid)) liquid substrate system (Sigma). The reaction is allowed to proceed for 30 minutes during which time the absorbance is read at 405 nm with an ELISA plate reader (Bio-Tek, Vt.). Sample concentrations of TF antigen are determined by interpolation against a standard curve performed with a series of known concentrations of biotinylated ASF. Values <15 ng/μL were considered as 0 (not detectable). Statistical significance between the values obtained for non-cancer and cancer ND values is determined by Student's t test.

The presence of TF correlates with disease presence. Background levels of TF antigen in healthy volunteers are very low. Statistical analyses demonstrated that the differences detected between cancer patients and healthy volunteers were significant, i.e., TF antigens are elevated in cancer patient ND (P=0.0007) and very low or non-detectable levels occur in healthy patients. ND samples were obtained from patients that had stage 0-4 disease with ductal and lobular location with and without lymph node metastases. There did not appear to be a correlation between disease stage or location and the expression level of either TF.

### uPA and PAI-1 Assays

Assays for uPA and PAI-1 are known in the art. (See, e.g., Qin et al. (2003) Ann Surg Oncol 10: 948-953; see also Sauter et al. (2008), Cancer Detection and Prevention 32: 149-155). In an exemplary embodiment, 100 μL of standards, ND samples and blanks are pipetted into the microplate wells that are coated with monoclonal antibodies respectively specific for uPA and PAI-1 and incubated overnight at 4° C. After washing (×4), enzyme-linked antibodies specific for each analyte are added to the wells and incubated for 1 hour at RT. The wells are washed again, diluted enzyme conjugate (streptavidin conjugated horseradish peroxidase) is pipetted into the wells, incubated (1 hour at RT), then washed again. Substrate reagent is added to each well, followed by a stop solution (0.5M sulfuric acid). Absorbance is measured with a microplate reader. Detection limits are 10 pg/mL for uPA and 50 pg/mL for PAI-1.

### Kits

The capture agent(s), labeled binding agent(s), revealing reagents, monocolonal antibodies, and/or standards for the conduct of the various capture immunoassays described herein may conveniently be supplied as kits which include the necessary components and instructions for the assays. Screening/diagnostic kits typically comprise one or more reagents that specifically bind to the target that is to be screened (e.g., ligands that specifically bind to TF-antigens). The reagents can, optionally, be provided with an attached label and/or affixed to a substrate (e.g., as a component of a protein array), and/or can be provided in solution. The kits can comprise nucleic acid constructs (e.g., vectors) that encode one or more such ligands to facilitate recombinant expression of such. The kits can optionally include one or more buffers, detectable labels or labeled binding agents, or other reagents as may be useful in a particular assay.

In addition, the kits optionally include labeling and/or instructional materials providing directions (i.e., protocols) for the practice of the methods described herein. Preferred instructional materials describe the detection of the targeted biomarkers in ND samples for the prediction, diagnosis, staging, and/or prognosis of breast cancer. While the instructional materials typically comprise written or printed materials, they are not limited to such. Any medium capable of storing such instructions and communicating them to an end user is contemplated. Such media include, but are not limited to electronic storage media (e.g., magnetic discs, tapes, cartridges, chips), optical media (e.g., CD ROM), and the like. Such media may include addresses to internet sites that provide such instructional materials.

A preferred kit includes one or more microtiter plates comprising wells, each coated with a specific antibody to one of the biomarkers, standard solutions for preparation of standard curve, a control for quality testing of the analytical run, the biomarkers conjugated to biotin or, alternatively, antibodies such as monoclonal antibodies, which are biotin-labelled and which can bind to each of the bound biomarkers, streptavidin-peroxidase enzyme, a substrate solution, a stopping solution, a washing buffer, and an instruction manual.

Having described the invention in detail, it will be apparent that modifications and variations are possible without departing the scope of the invention defined in the appended claims. Furthermore, it should be appreciated that all examples in the present disclosure are provided as non-limiting examples.

### EXAMPLE

The following non-limiting example is provided to further illustrate the present invention. It should be appreciated by those of skill in the art that the techniques disclosed in the example that follows represent an approach the inventors have found function well in the practice of the invention, and thus can be considered to constitute an examples of the best modes for its practice. However, those of skill in the art should, in light of the present disclosure, appreciate that many changes can be made in the specific embodiments that are disclosed and still obtain a like or similar result without departing from the spirit and scope of the invention.

### I. Methods

### A. Patients and Samples

After receiving Institutional Review Board approval, and informed consent, participants were prospectively enrolled and 79 ND samples were collected in Grand Forks, N. Dak., at the University of North Dakota, in Columbia, Mo., at the Ellis Fischel Cancer Center and in London, UK, at the Royal Marsden Cancer Center. Details of TF sample collection and analysis were previously reported [Deutscher (2010)]. Among the 124 ND samples collected in the TF report there was sufficient remaining NAF to analyze both uPA and PAI-1 in 24 and uPA alone in an additional 11 samples. Criteria for enrollment were the same in the current as in Deutscher (2010).

A subject was classified as postmenopausal if at least one year had passed without a menstrual period or she had undergone bilateral oophorectomy prior to enrollment. Women who had undergone hysterectomy without bilateral oophorectomy were considered postmenopausal if they were over 50 years old. If follicle stimulating hormone (FSH) levels were available, a level of 34 mIU/mL or greater was used to classify women as postmenopausal. All ND samples were collected prior to excisional biopsy or mastectomy. Comparisons of uPA, PAI-1 and TF were based on the histopathologic findings in the clinical report.

### B. Sample Collection

ND (1-10 μL) was obtained from the breast with a lesion prior to surgery. Lesions included women with 1) pathologic nipple discharge (PND); 2) a suspicious lesion identified on imaging, be it mammogram, ultrasound or breast magnetic resonance imaging; and/or 3) a palpable lesion that was not a simple cyst. Samples were collected as described previously [Sauter (1997)]. Briefly, after informed consent was obtained, ND fluid was aspirated using a breast pump (NAF) or collected after the participant massaged her breast (PND). Samples were collected into capillary tubes, the volume of NAF measured and stored at −80° C. until use.

### C. Preparation

The portion of the capillary tube containing the sample was introduced into a 1.7 mL Eppendorf tube and 100 μL of a 0.1 mol/L solution of sodium bicarbonate (pH 7.8) added. The capillary tube was then crushed with a glass rod and the mixture vortexed to disperse the sample. The crushed capillary tube was left in the bicarbonate buffer overnight at 4° C., and the mixture then centrifuged (14,000 g, 5 min) and the supernatant used without further dilution.

### D. Analysis

uPA and PAI-1. ELISA kits for uPA and PAI-1 were obtained from American Diagnostica, Inc. (Greenwich, Conn.). Levels of these two markers in ND samples were determined according to the manufacturer's instructions. Briefly, 100 uL of standards, samples and blanks were pipetted into microplate wells coated with monoclonal antibodies respectively specific for uPA and PAI-1 and incubated overnight at 4° C. After washing (×4), enzyme-linked antibodies specific for each analyte were added to the wells and incubated for 1 h at room temperature. The wells were washed again, diluted enzyme conjugate (streptavidin conjugated horseradish peroxidase) was pipetted into the wells, incubated (1 h, RT), then washed again. Substrate reagent was added to each well, followed by a stop solution (0.5M sulfuric acid). Absorbance was measured with a microplate reader. Detection limits were 10 pg/mL for uPA and 50 pg/mL for PAI-1.

TF. The measurement of this carbohydrate was previously reported [Deutscher (2010)].

### E. Statistical Analysis

Biomarker levels for uPA and PAI-1 in the ND samples were heavily skewed and not normally distributed. Therefore biomarker levels were described and analyzed using medians and log-transformed (Log 10) means to achieve normal distributions. A Shapiro-Wilk test was used to determine the goodness-of-fit of the data to a normal distribution. None of the log-transformed variables were significantly different from a normal distribution. Unpaired t-tests were calculated to determine significant differences between log 10 means of the biomarkers. Logistic regression analyses are based on log 10 transformed data. Three logistic regression models were used to examine the effects of uPA and PAI-1 in predicting (a) cancer vs. no cancer diagnosis; (b) cancer vs. benign diagnosis; and (c) abnormal vs. benign diagnosis. Various demographic factors, include age, menopausal status, and hormone replacement, were included in the logistic models as covariates. Because menopausal status was a near significant predictor in all three overall logistic models, follow-up logistic models were conducted separately for pre- and for post-menopausal women, controlling for age. ROC curves were calculated based on the logistic regression results for the premenopausal women's data, and AUC values are presented. The optimal subset of all variants, uPA, PAI-1, TF, Tn, and covariates was selected to achieve the best AUC value.

### II. Results

### A. Recruitment

NAF was successfully collected in 90% (76/84) and PND in 100% (3/3) women. Among the 79 women evaluated for uPA and PAI-1 expression, 41 (51.9%) were postmenopausal. Age ranged from 21 to 82 years, with a median age in pre- and postmenopausal women of 44 and 61, respectively. 25 women took hormone replacement therapy (HRT) at one point in their lives, with 2 at the time of NAF collection. Among the 35 women evaluated for TF, uPA+/−PAI-1 expression, 17 (48.6%) were postmenopausal. Age ranged from 26 to 77 years, with a median age in pre- and postmenopausal women of 43.5 and 61 years, respectively. 11 women took hormone replacement therapy (HRT) at one point in their lives, with 3 at the time of NAF collection.

B. Fluid Volume and Biomarker Expression not Different Among Women with Vs. Those without PND

ND volume ranged from 4 to 521 microliters, with a mean of 60.9 and median of 29 microliters. ND volume did not vary by pathologic diagnosis nor by fluid type (NAF vs. PND). It was first determined if uPA and PAI-1 expression in women requiring diagnostic biopsy due to PND differed from expression in women requiring biopsy who did not have PND. Results of logistic regression analyses conducted using samples from women with PND and NAF showed that fluid type did not affect expression levels, with p>0.5 for all biomarkers. Therefore, all reported analyses include both PND and NAF samples (N=79).

C. uPA Concentration is Associated with Breast Atypia and Cancer

uPA concentration was higher (Table 1) in women with cancer (DCIS or invasive) than in women with 1) no cancer: atypia or benign pathology (p=0.023), and 2) benign pathology (p=0.025). uPA was also higher in women with abnormal (atypia or cancer) than benign pathology (p=0.050). PAI-1 was higher in women with cancer (DCIS or invasive) than in women with benign pathology (p=0.037).

TABLE 1
Univariate analyses of uPA and PAI-1 expression
(ng/mL) in women requiring breast surgery

| A. Cancer/no cancer | Overall | No cancer¹ | Cancer¹ | P value |
|---|---|---|---|---|
| All Subjects (N) | 79 | 46 | 33 | |
| uPA Mean (SD) | 2.4 (.90) | 2.2 (.84) | 2.7 (.92) | .023 |
| Median | 2.4 | 2.2 | 2.6 | |
| PAI-1 Mean (SD) | 3.7(1.5) | 3.4 (1.4) | 4.2 (1.6) | .064 |
| Median | 3.5 | 3.3 | 4.1 | |
| Premenopausal (N) | 38 | 23 | 15 | |
| uPA Mean (SD) | 2.5 (.93) | 2.2 (.82) | 2.9 (.93) | .018 |
| Median | 2.5 | 2.3 | 2.7 | |
| Postmenopausal (N) | 41 | 23 | 18 | |
| PAI-1 Mean (SD) | 4.0 (1.6) | 3.5 (1.6) | 4.6 (1.4) | .025 |
| Median | 3.8 | 3.3 | 4.3 | |
| B. Cancer/benign | Overall | Benign¹ | Cancer¹ | P value |
| All Subjects (N) | 72 | 39 | 33 | |
| uPA Mean (SD) | 2.4 (.93) | 2.2 (.89) | 2.7 (.92) | .025 |
| Median | 2.4 | 2.2 | 2.6 | |
| PAI-1 Mean (SD) | 3.7 (1.5) | 3.4 (1.4) | 4.2 (1.6) | .037 |
| Median | 3.4 | 3.1 | 4.1 | |
| Premenopausal (N) | 37 | 22 | 15 | |
| uPA Mean (SD) | 2.4 (.95) | 2.2 (.84) | 2.9 (.93) | .017 |
| Median | 2.5 | 2.2 | 2.7 | |
| Postmenopausal (N) | 35 | 17 | 18 | |
| PAI-1 Mean (SD) | 4.0 (1.7) | 3.4 (1.7) | 4.6 (1.4) | .033 |
| Median | 3.8 | 2.8 | 4.3 | |
| C. Abnormal/benign | Overall | Benign¹ | Abnormal¹ | P value |
| All Subjects (N) | 79 | 39 | 40 | |
| uPA Mean (SD) | 2.4 (.90) | 2.2 (.89) | 2.6 (.88) | .050 |
| Median | 2.4 | 2.2 | 2.5 | |
| PAI-1 Mean (SD) | 3.7 (1.5) | 3.4 (1.4) | 4.0 (1.6) | .060 |
| Median | 3.5 | 3.1 | 3.9 | |
| Premenopausal (N) | 38 | 22 | 16 | |
| uPA Mean (SD) | 2.5 (.93) | 2.2 (.84) | 2.9 (.91) | .018 |
| Median | 2.5 | 2.2 | 2.7 | |
| Postmenopausal (N) | 41 | 17 | 24 | |
| PAI-1 Mean (SD) | 4.0 (1.6) | 3.4 (1.7) | 4.4 (1.3) | .052 |
| Median | 3.8 | 2.8 | 4.1 | |

¹No cancer = normal, hyperplasia, and atypical hyperplasia; Cancer = ductal carcinoma in situ and invasive cancer; Benign = normal, and hyperplasia; Abnormal = atypical hyperplasia, ductal carcinoma in situ and invasive cancer. Means and medians reflect log10 values for each marker. For all subjects, all p values are provided regardless of the result. For pre- and postmenopausal groupings, only results which were significant or approached significance (p < .065) were included.

### D. uPA and PAI-1 Expression Based on Menopausal Status

Univariate analysis indicated that uPA concentration was more predictive of disease in premenopausal women, and PAI-1 in postmenopausal women (Table 1). uPA concentration was higher in premenopausal women with cancer (DCIS or invasive) than in women with 1) no cancer (p=0.018), and 2) benign pathology (p=0.017), and in women with abnormal pathology than those with benign pathology (p=0.018). PAI-1 concentration was higher in postmenopausal women with cancer (DCIS or invasive) than in women with 1) no cancer (p=0.025), and 2) benign pathology (p=0.033).

In 79 ND samples, ROC curves were generated to determine how well information on uPA and age predicted if a premenopausal woman had breast atypia or cancer. Three comparisons: cancer vs. no cancer, cancer vs. benign pathology, and abnormal vs. benign pathology were conducted. The AUC values ranged from 0.83-0.87 (Table 2), and were better than those for postmenopausal women.

TABLE 2
AUC values for disease prediction
considering uPA, PAI-1, TF and age¹

| | | | | AUC | |
|---|---|---|---|---|---|
| | | | CA/no | CA/ | Abnormal/ |
| Biomarker | Group | N¹ | CA² | benign² | benign |
| uPA + PAI-1 | All | 79 | .72 | .75 | .74 |
| uPA³ | Pre | 38 | .87 | .83 | .86 |
| TF | Post | 72 | .81 | .83 | .83 |
| TF + uPA | All | 35 | .84 | .92 | .91 |
| TF + uPA + PAI-1 | All | 24 | 1.0 | 1.0 | .97 |

¹AUC: area under the receiver operating curve; uPA: urinary plasminogen activator; PAI (uPA inhibitor)-1, TF: Thomsen-Friedenreich antigen; N = number or sample size
²Cancer = ductal carcinoma in situ (DCIS), and invasive cancer; Benign = normal and hyperplasia; Abnormal = atypical hyperplasia, DCIS, and invasive cancer. Age is in all three models.
³AUC values for postmenopausal women were lower.

### E. The Combination of TF, uPA and PAI-1 are Highly Predictive of the Presence of Breast Cancer

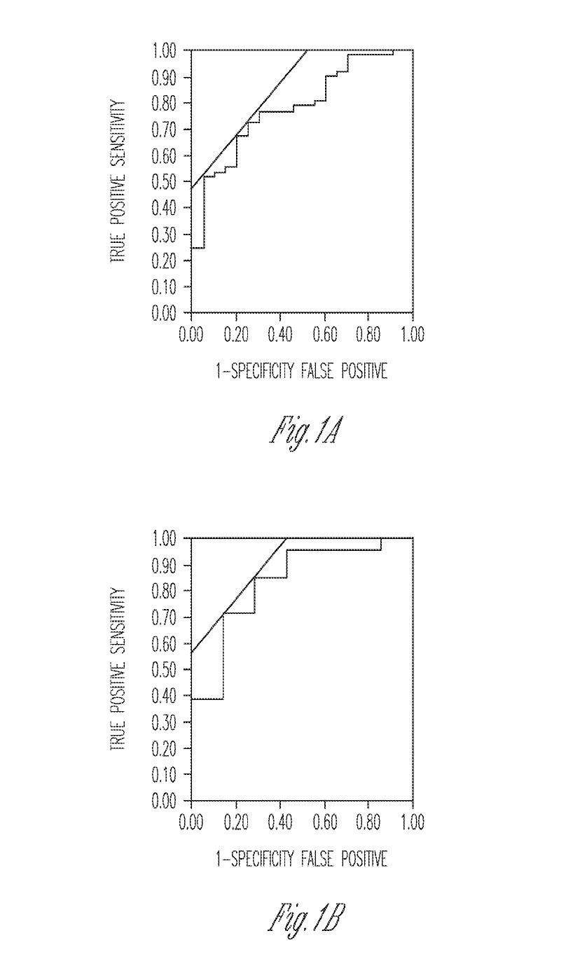

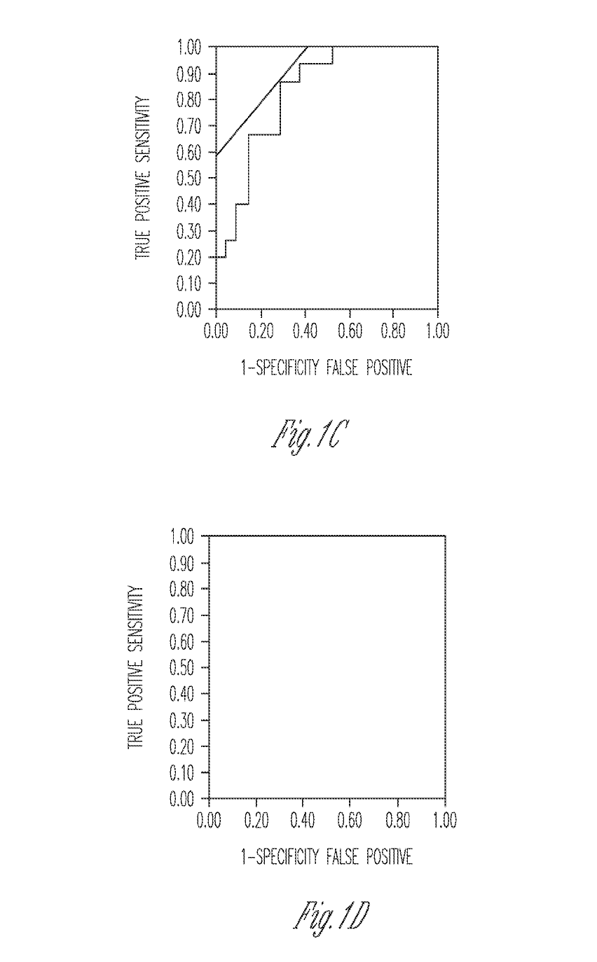

Among the 79 samples evaluated for uPA+PAI-1 expression, the AUC for age alone was 0.62. The AUC for uPA, PAI-1 and age ranged from 0.72 to 0.75 when predicting disease in all women, whereas the AUC for uPA and age in premenopausal women only was higher (0.83-0.87). The levels of uPA and PAI-1 were measured in NAF collected contemporaneously with that used to measure TF Deutscher (2010). For all of these analyses, age was in the predictive model. TF+uPA predicted disease in both pre- and postmenopausal women with 84-92% accuracy (FIG. 1C). When TF, uPA, and PAI-1 were combined, the predictive ability approached 100% (Table 2, FIG. 1D).

### III. Discussion

Carbohydrate biomarkers have not been investigated as extensively as proteins in NAF or other bodily fluids. Levels of TF were reported as measured by direct immunoassay in 124 ND samples, including 52 from pre- and 72 from post-menopausal women with a suspicious breast lesion which required biopsy [Deutscher (2010)]. Age alone in this cohort provided an AUC of 0.69. TF and age, predicted the presence of atypia and cancer in the postmenopausal group with an AUC of 0.83.

In the present analysis of 79 ND samples (different than the 124) with similar enrollment criteria, uPA was highly predictive of breast cancer in premenopausal women (0.83-0.87), but less so in postmenopausal women. It was determined that two markers were better than one in predicting disease in all women, with TF+uPA having an AUC of 0.84-0.92. When TF, uPA, and PAI-1 were combined, the AUC approached 1.0%.

Previously, it was reported that the expression of some ND cancer prediction markers varied based on whether or not the subject requiring surgery did or did not have PND (Sauter, Cancer Detect. Prey. (2007) 31, 50-58). TF concentration was not influenced by the presence or absence of PND [Deutscher (2010)]. Therefore, it was determined if uPA and PAI-1 concentrations differed based on whether the sample was PND or NAF. Since a concentration difference was not noted, all analyses included both PND and NAF samples.

All publications cited herein are incorporated by reference herein as though fully set forth. Whereas, the present invention has been described in relation to the drawings attached hereto, it should be understood that other and further modifications, apart from those shown or suggested herein, may be made within the spirit and scope of this invention.

I claim:

1. A method for diagnosing breast cancer in a human subject suspected of having breast cancer, comprising the steps of:
collecting nipple discharge from said subject;
analyzing said discharge for the level of (a) the TF carbohydrate biomarker, (b) the uPA protein biomarker and (c) the PAI-1 protein biomarker; and
determining the presence or absence of breast cancer based on the results of said analysis.

2. A method for diagnosing breast cancer in a human subject suspected of having breast cancer comprising the steps of:
(i) analyzing a sample of nipple discharge from said subject to determine the level of (a) the TF carbohydrate biomarker, (b) the uPA protein biomarker and (c) the PAI-1 protein biomarker;
(ii) determining the presence or absence of breast cancer based on the results of the analysis; and
(iii) informing the subject or his/her physician of the presence or absence of breast cancer.

3. The method of claim 1 wherein the subject is a woman.

4. The method of claim 2 wherein the subject is a woman.

5. The method of claim 1, 2, 3 or 4 wherein said determination is based on logistic regression and takes into account the age of said subject as well as the levels of said biomarkers.

6. The method of claim 1, 2, 3 or 4 wherein said nipple discharge is nipple aspirate fluid.

7. The method of claim 1, 2, 3 or 4 wherein said nipple discharge is spontaneous discharge.

8. The method of claim 7 in which said spontaneous discharge is selected from the group consisting of physiologic discharge and pathologic discharge.

9. The method of claim 1, 2, 3 or 4 wherein said determination is positive where each of said biomarkers are elevated above the level of said biomarker present in subject suspected of having cancer but not having cancer and wherein said determination is negative where at least one of said biomarkers is not elevated above said level.

10. The method of claim 9 further comprising the step of performing a biopsy on said woman wherein said determination is positive.

11. The method of claim 1, 2, 3 or 4 wherein said analysis comprises an immunoassay.

12. The method of claim 11 wherein said analysis comprises detection with monoclonal antibodies specific to each of said biomarkers.

13. The method of claim 3 or 4 wherein said subject is premenopausal.

14. The method of claim 3 or 4 wherein said subject is postmenopausal.

15. A kit for detection of carbohydrate and protein biomarkers in nipple discharge or a derivative thereof, said kit comprising:
(a) a capture agent that binds specifically to TF;
(b) a capture agent that binds specifically to uPA;
(c) a capture agent that binds specifically to PAI-1;
(d) a labeled binding agent that binds to bound TF;
(e) a labeled binding agent that binds to bound uPA;
(f) a labeled binding agent that binds to bound PAI-1; and
(g) instructions for use of the kit in a method according to claim 1 or 2.
